(19) United States
(12) Patent Application Publication
(10) Pub. No.: US 2025/0381069 A1
(43) Pub. Date: Dec. 18, 2025

(54) INTERMEDIATE STORAGE UNIT FOR CYCLICAL POWER SMOOTHING AND OPHTHALMOLOGICAL LASER THERAPY SYSTEM HAVING AN INTERMEDIATE STORAGE UNIT

(71) Applicant: Carl Zeiss Meditec AG, Jena (DE)

(72) Inventors: Gregor STOBRAWA, Jena (DE); Wolf WEIMER, Jena (DE); Torsten FEIGE, Jena (DE); Andreas KNAUER, Jena (DE); Tobias KRAUSE, Konigshain (DE)

(21) Appl. No.: 18/698,295

(22) PCT Filed: Oct. 4, 2022
(86) PCT No.: PCT/EP2022/077510

§ 371 Date: Feb. 25, 2025

(30) Foreign Application Priority Data
Oct. 5, 2021 (DE) 10 2021 211 227.7

(51) Int. Cl. A61F 9/008; A61B 17/00

(52) U.S. Cl. CPC A61F 9/008; A61B 2017/00734

(57) ABSTRACT

Please substitute the following amended Abstract for the Abstract as currently pending (deleted matter is shown by strikethrough and added matter is shown by underlining):

When using an ophthalmological laser therapy system, it is advantageous if the system can be installed globally without a person skilled in the art and flexibly in terms of space. This demands operability at a power consumption of only 1600 watts. The present invention therefore ensures operability even when power peaks occur. An intermediate storage unit for an ophthalmological laser therapy system with power consumption of up to 1600 watts, the system having an electrical load element. Also, an ophthalmological laser therapy system with power consumption of up to 1600 watts, the system having an electrical load element and an intermediate storage unit.

### CLAIM TO PRIORITY

This application is a National Stage entry of Application PCT/EP2022/077510, filed Oct. 4, 2022, and claims priority from DE Patent Application No. 10 2021 211 227.1, filed Oct. 5, 2021, each of which are incorporated by reference in their entireties in this application.

### TECHNICAL FIELD

The present invention relates to an intermediate storage unit for an ophthalmological laser therapy system with a power consumption of up to 1600 W, which system has an electrical load element. The invention furthermore relates to an ophthalmological laser therapy system having an intermediate storage unit.

### BACKGROUND

Ophthalmological laser therapy systems are complex electromechanical arrangements having various components. These components are electrical consumers that constitute a corresponding electrical load. Such load elements can be for example computers, user interfaces such as monitors, control devices or actuators for mechanical movements, and also the laser source itself and the control device thereof. An ophthalmological laser therapy system typically has a unit for the electromotive adjustment of the laser beam and thus of the interaction location: this unit also constitutes an electrical load element. All these components typically consume electrical power during operation of the ophthalmological laser therapy system, the power consumption being able to be divided into a portion representing a quasi-constant basic consumption, and a variable portion. The variable portion of the power consumption may increase significantly during a laser treatment and thus cause considerable power fluctuations, for example. Specific requirements made of an energy supply of the load elements arise as a result of severe power fluctuations.

When designing an ophthalmological laser therapy system, it should be taken into consideration that the available electrical power is limited, in principle. The location at which the laser system is intended to be installed is typically the limiting factor here. More specifically, fuse values, cable cross sections and connector types influence the maximum available electrical power. Furthermore, the available power is limited by the local electrical power supply system voltage (in volts, V) of the local power supply system, which globally is between 100 V (for example in Japan) and 240 V (as in Europe, for instance).

In order to be able to sell and use an electrical device globally, without having to take account of particular local characteristics of the power supply, wide-range power supply units and interchangeable power supply system cables with a country-specific connector are typically used. The use of wide-range power supply units ensures that the device will function globally since the energy supply of the device remains guaranteed despite provision of different local power supply system voltages. A wide-range power supply unit should thus be understood to mean a power supply unit having a wide-range input, via which the power supply unit is operable with all globally customary power supply system voltages. In this way, simple installation of the device is possible globally, without the installation necessitating a person skilled in the art for electrical switchgear. This is advantageous particularly if one in the same device is intended to be used at different locations. In order to be able to attain a maximum input power for the device, power supply system cables which allow a maximum current should be used. If standardized power supply system cables are used, this can be made possible for example with the connector type IEC-60320 C19/C20 (connector for non-heating devices for 16amperes, A). If the device is intended to be able to be used globally, the resulting minimum of a maximum power consumption (in watts, W) is a value of 100 V*16 A=1600 W. Taking into consideration 10% tolerance in the power supply system voltage, necessary compliance with the maximum current of 16 A (for fuse tripping, owing to cable certification, . . . ) and a typical efficiency of the power supply unit of approximately 80-90%, the resulting used electrical power for the device is approximately 1300 W. With a large phase angle, a further reduction of the used power may occur.

In the case of medical devices, and in particular an ophthalmological laser therapy system, it is advantageous to dispense with the use of non-demountable electrical connections (e.g. for three-phase current), in order that the medical device can be installed globally and spatially flexibly (e.g. at different locations in a clinic) without a person skilled in the art. It is furthermore advantageous for the medical device (and in particular the power supply unit thereof) not to be manufactured in a country-specific manner with a power supply system cable non-detachably connected to the device, since this can lead to increased production and/or service expenditure. Under these constraints, it is therefore desirable to implement a medical device in such a way that it is fully functional with a power consumption of only 1600 W.

More specifically, this means that a power budget for the various components (electrical load elements) of the medical device must be allocated in order to meet the abovementioned condition of a maximum power consumption. The electrical load elements, in particular those having a high power demand, in the case of an ophthalmological laser therapy system, may be a laser source for generating a therapy beam, a scanning unit for beam deflection of the laser beam, a computer, a monitor or, for example, an adjusting unit for aligning the patient in relation to the laser system.

On account of the limited maximum power consumption, ensuring the functionality of the medical device despite power fluctuations that may occur during the operation thereof is a particular challenge. The document US 2008/0269729 A1 proposes a method and a device that ensure safe use of an ophthalmological laser therapy system in the event of an extreme power fluctuation, namely in the event of failure of the power supply of the system. For this purpose, the laser system has a rechargeable battery, the capacity of which is high enough to ensure that the laser therapy treatment is carried out in the case of an interruption of the power supply. However, the cited document does not disclose a solution as to how a functionality of the laser system can be ensured upon the occurrence of power spikes that may occur during the actual therapeutic intervention, for example.

Therefore, it is an object of the present invention to ensure the functionality of an ophthalmological laser therapy system having a limited power consumption if power spikes occur.

### SUMMARY OF THE INVENTION

A first aspect of the invention relates to an intermediate storage unit for an ophthalmological laser therapy system.

An ophthalmological laser therapy system is configured to carry out an ophthalmological therapy by application of laser radiation when used by a user (operating physician, surgeon, operator). Here, an ophthalmological therapy should be understood to mean any therapy in which ocular tissue, for example the cornea, is modified. In particular, the ophthalmological therapy includes corresponding laser-surgical interventions, in which ocular tissue is “cut” (for example, a lenticule or flap) by photodisruption by application of a laser, a pulsed laser such as a femtosecond or excimer laser for example, in which a region of an ocular tissue is ablated by an ablation effect or in which ocular tissue is “adhesively bonded” to itself by a coagulation effect, or in which the refractive index of the material, that is to say of an ocular tissue or else an implant, is modified by the laser radiation.

In the context of the present disclosure, an ophthalmological laser therapy system is also referred to as a “laser system”.

In this case, the ophthalmological laser therapy system has a power consumption of up to 1600 W. For this purpose, the laser system can have a wide-range power supply unit and a (interchangeable) power supply system cable with a country-specific connector. The power supply system cable can be for example a standardized power supply system cable designed for a current of 16 A. Taking into consideration tolerances in the power supply system voltage and a typical efficiency of power supply units, used electrical power of approximately 1300 W is thus available to the laser system.

Furthermore, the ophthalmological laser therapy system has an electrical load element. In the context of the disclosure, an electrical load element is also referred to simply as “load element”. In this case, the load element can have one or more electrical components. In other words, an electrical load element constitutes a consumer of electrical energy or has one or a plurality of such consumers.

An electrical load element can be for example a laser unit for providing therapeutic laser radiation. It can also be a control unit (also called control device) for controlling electrical load elements of the laser system. It can be a computing unit (computer with CPU and memory) or a monitor. The laser system can also have a positioning unit, which typically comprises actuators, for aligning a patient in relation to the laser system as an electrical load element. The load element can also be a beam offset unit for the electromotive, spatial adjustment of the laser beam and thus of the interaction location (in two or three spatial dimensions). For this purpose, the beam offset unit can have one or more scanners and also a scanner controller.

According to the invention, the intermediate storage unit is configured to provide cyclically an energy of at least 30 mJ (millijoules), preferably at least 40 mJ, particularly preferably at least 50 mJ, for the electrical load element. Cyclical provision should be understood here to mean that an energy is available for consumption by the load element a number of times (at least 10 times, for example at least 100 times, in another example at least 1000 times). In this case, the frequency of the provision of the energy need not be constant over the number of provisions. The frequency can change for example over time continuously, linearly or with a (integer or non-integer) power during the time.

The cyclical provision of energy enables cyclically occurring power spikes as a result of power requisitioning by the load element of the laser system to be absorbed, buffered or smoothed, without the maximum power consumption of the laser system being exceeded. As a result, the smooth progression of a laser therapy intervention can be ensured even for such power spikes.

In one example configuration, the intermediate storage unit is configured to provide cyclically an energy of up to 220 mJ, for example up to 200 mJ, in another example up to 180 mJ, for the electrical load element.

In accordance with a further configuration of the intermediate storage unit, the latter is configured to provide the energy cyclically at a frequency of at least 20 Hz, for example at least 40 Hz, in another example at least 60 Hz. Additionally or alternatively, the intermediate storage unit is configured to provide the energy cyclically at a frequency of up to 6 kHz, for example up to 5.5 kHz, in another example up to 5 kHz.

If the electrical load element is the beam offset unit, then the latter could for example deflect the laser radiation cyclically at least 10 Hz (or respectively at least 20 Hz or 30 Hz) and/or at up to 3 kHz (or respectively 2.75 kHz or 2.5 kHz); the halving of the frequencies results from the fact that two power spikes may occur within a scanning period of the beam offset unit.

By virtue of the aforementioned frequency ranges, power spikes caused for example by the beam offset unit during the scanning of the laser beam in the eye—in order to cut a lenticule or a flap, for example—can advantageously be absorbed or buffered in such a way that the maximum power consumption of the laser system is not exceeded. As a result, the smooth progression of a laser therapy intervention can be ensured even for such power spikes.

In accordance with one example configuration of the intermediate storage unit, the latter has a capacitor and/or a battery. In this way, rapid charge-reversal processes are made possible, i.e. a rapid delivery of energy, as is required for example during cyclical provision of energy in the kilohertz range. Even high power spikes can be smoothed in this way. For this purpose, a capacitor for example has a high capacitance in order to be able to ensure a high current flow for a short period. The capacitor can be an electrolytic capacitor or a so-called super-cap. A battery for example has a low internal resistance. The latter can be less than 0.1 ohm or up to 0.001 ohm, for example. In the case of batteries having a low internal resistance, the capacities required are not as high since the voltage does not collapse as rapidly under load. The battery can be for example a so-called high-current rechargeable battery (e.g. LiPo rechargeable battery).

In one example configuration, the intermediate storage unit is characterized in that the cyclical provision of energy is effected for a load element having a cyclical power demand. During a laser therapy treatment, a cyclical power demand can lead to cyclical power spikes. A load element having a cyclical power demand can be for example the laser unit for providing therapeutic laser radiation. The load element can also be the beam offset unit for the electromotive, spatial adjustment of the laser beam and thus of the interaction location (in two or three spatial dimensions). For this purpose, the beam offset unit can have one or more scanners and also a scanner controller. Advantageously, the intermediate storage unit is configured to provide the energy for the load element at a frequency corresponding to the frequency of the cyclical power demand.

A second aspect of the invention relates to an ophthalmological laser therapy system. The ophthalmological laser therapy system has a power consumption of up to 1600 W, and also an electrical load element. According to the invention, the ophthalmological laser therapy system furthermore comprises an intermediate storage unit according to one of the configurations explained above.

In one preferred configuration of the ophthalmological laser therapy system, the latter has at least one further electrical load element and also an energy supply unit. In this case, the energy supply unit is electrically connected to the electrical load element and the at least one further electrical load element in such a way that a supply with energy is made possible. The energy supply unit distributes the energy between the at least two load elements, as it were. In this case, the entire consumed power of the laser system of 1600 W can be available to the energy supply unit.

According to example embodiments of the invention, the intermediate storage unit is electrically connected to the energy supply unit and the electrical load element in such a way that the energy is provided cyclically only for the electrical load element. In other words, the required energy is provided cyclically only for the electrical load element whose cyclical power spikes are intended to be buffered, while further electrical load elements are excluded from this. In this way, it can advantageously be ensured that “smoothing” of power spikes of the electrical load element does not adversely affect further electrical load elements. In a power supply system of the laser system, the required power is thus buffered as it were by the intermediate storage unit at the location where power is drawn variably.

In one variant of the ophthalmological laser therapy system, for example according to one of the configurations described above, having a power consumption of up to 1600 W, this system has at least two electrical load elements, a control unit and for example an intermediate storage unit according to one of the abovementioned configurations. In this case, the control unit is configured according to the invention in such a way that the electrical load elements can be switched on and off sequentially. Switching the load elements on and off sequentially makes it possible to avoid an accumulation of power spikes that arises as the sum or superposition of power spikes of the individual load elements. Furthermore, in this way, electrical load elements that are not required during a phase of the ophthalmological intervention, for example, can be switched off in order to temporarily reduce the total power required.

In accordance with one configuration of the ophthalmological laser therapy system, the latter is characterized in that it has an energy supply unit configured as a wide- range power supply unit. Furthermore, the ophthalmological laser therapy system comprises an interchangeable power supply system cable, via which the energy supply unit can be electrically connected to a local power supply system. This makes it possible to be able to sell and use the ophthalmological laser therapy system globally, without having to take account of particular local characteristics of the power supply. At the same time, by way of the intermediate storage unit, it is ensured that power spikes during operation of the device can be buffered, even though the power consumption is only up to 1600 W.

It goes without saying that the features mentioned above and the features yet to be explained hereinafter can be used not only in the specified combinations but also in other combinations or on their own, without departing from the scope of the present invention.

### BRIEF DESCRIPTION OF THE DRAWINGS

The invention is explained in greater detail below for example with reference to the accompanying drawings, which also disclose features essential to the invention. In the figures:

FIG. 1 is a schematic illustration of an exemplary ophthalmological laser therapy system with an intermediate storage unit;

FIG. 2 is an illustration of the schematic set-up of an ophthalmological laser therapy system according to the invention with an intermediate storage unit.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

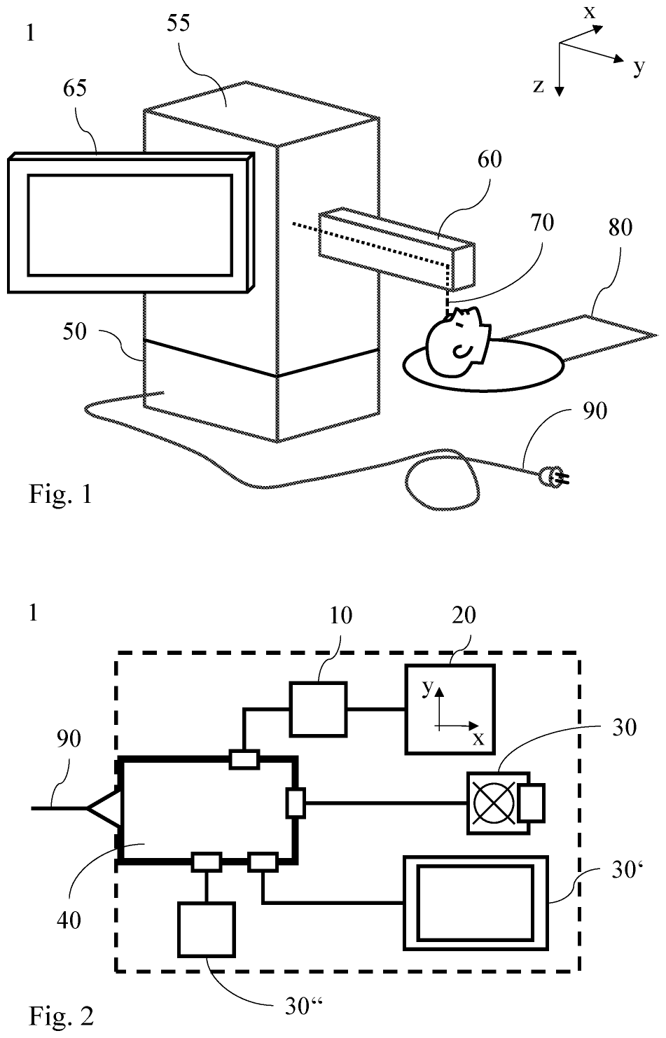

FIG. 1 is a schematic illustration of an exemplary ophthalmological laser therapy system 1.

The example of the ophthalmological laser therapy system 1 includes a device base 50 and a device head 55 that is adjustable on this device base 50 in terms of its height above a ground plane, that is to say the z-direction, and in terms of its position in the plane, that is to say in the x-and y-directions. In the example shown, the device head 55 comprises a laser unit (not illustrated), here a femtosecond laser source, required to generate a corresponding pulsed laser beam. The therapeutic laser radiation 70 generated by the laser source is guided via a laser arm 60 to the therapy location in a patient's eye. The therapeutic laser radiation 70 is represented by a dotted line within the laser system 1, and by a dashed line outside that. For the electromotive, spatial adjustment of the laser beam and thus of the interaction location (therapy location in the patient's eye), the device head 55 has a beam offset unit (not depicted). In the example shown, the beam offset unit has a pair of scanners that allow a deflection of the therapeutic laser radiation 70 at the therapy location in the x-y-plane.

The patient can be aligned in relation to the ophthalmological laser therapy system 1 not just by way of a movement of the device head 55. In addition, the laser system has a patient couch 80 as positioning unit, the patient couch comprising actuators and likewise allowing a movement of the patient relative to the laser system 1.

Furthermore, the ophthalmological laser therapy system 1 has a monitor 65. The latter allows the user of the laser system 1 to monitor and control a laser therapy treatment. The operating units (key board, joystick) additionally required for control are not depicted.

The ophthalmological laser therapy system 1 is supplied with energy via a power supply system cable 90. The latter is a standardized power supply system cable with a connector of the connector type IEC-60320 C19/C20. The laser system I can be provided with a current of 16 A via the power supply system cable 90. For this purpose, the power supply system cable 90 is connected to a wide-range power supply unit (not depicted). In this case, the connection is configured so as to enable the power supply system cable 90 to be exchanged for a different power supply system cable 90 with a different connector (without a service technician). The wide-range power supply unit can process voltages of 100 V to 240 V and ensure an energy supply of the various load elements (consumers) according to the voltage respectively required. If a voltage of 100 V is provided via the local power supply system, then the power consumption of the ophthalmological laser therapy system 1 is up to 1600 W.

According to the invention, the laser system 1 has an intermediate storage unit (not depicted). The latter is electrically connected firstly to the wide-range power supply unit and secondly to the beam offset unit. The beam offset unit here is the electrical load element which, during a laser therapy treatment, cyclically (depending on the oscillation frequency of the scanners) has a high power demand and thus has power spikes.

According to the invention, the intermediate storage unit is configured to smooth these cyclically occurring power spikes by providing energy.

FIG. 2 is an illustration of the schematic set-up of an ophthalmological laser therapy system I according to the invention with an intermediate storage unit 10. The laser system 1 is electrically connected to the local power supply system (not depicted) via a power supply system cable 90. The releasable electrical connection is represented by a triangular symbol on the power supply system cable 90. The power supply system cable 90 feeds energy to the energy supply unit 40, configured as a wide-range power supply unit. The latter supplies a plurality of consumers of the laser system 1 with energy. The beam offset unit is an electrical load element 20 which, during a laser therapy treatment, cyclically has a high-power demand and thus has power spikes. In order to smooth these power spikes, the electrical load element 20 is electrically connected to the intermediate storage unit 10, which is in turn electrically connected to the energy supply unit 40. According to example embodiments of the invention, the intermediate storage unit is configured to smooth these cyclically occurring power spikes.

In addition, the ophthalmological laser therapy system 1 has further electrical load elements 30, 30′ and 30″, which do not cause cyclical power spikes that need to be smoothed during operation. These elements here are a laser unit, a monitor and a control unit, respectively. In the illustrated arrangement, the intermediate storage unit 10 is electrically connected to the energy supply unit 40 and the electrical load element 20, which can cause cyclical power spikes during operation, in such a way that the energy is provided cyclically only for the load element 20, while the further electrical load elements 30, 30′ and 30″ are not influenced by the smoothing of the power spikes.

In this case, the features of the invention mentioned above and described in various exemplary embodiments can be used not only in the exemplary combinations indicated, but also in other combinations or by themselves, without departing from the scope of the present invention.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features: rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims. it is expressly intended that the provisions of 35 U.S.C. § 112(f) are not to be invoked unless the specific terms “means for” or “step for” are recited in a claim.

1.-9. (canceled)

10. An intermediate storage unit for an ophthalmological laser therapy system with a power consumption of up to 1600 W, the system comprising an electrical load element, the intermediate storage unit being configured to cyclically provide an energy of at least 30 mJ, for the electrical load element.

11. An intermediate storage unit as claimed in claim 10, wherein the intermediate storage unit is configured to cyclically provide an energy of at least 40 mJ, or at least 50 mJ, for the electrical load element.

12. The intermediate storage unit as claimed in claim 10, wherein the intermediate storage unit is configured to cyclically provide an energy selected from a group consisting of up to 220 mJ, up to 200 mJ, up to 180 mJ, for the electrical load element.

13. The intermediate storage unit as claimed in claim 10, wherein a cyclical provision is effected at a frequency selected from a group consisting of at least 20 Hz, at least 40 Hz, and at least 60 Hz, wherein the cyclical provision is effected at a frequency selected from a group consisting of up to 6 kHz, up to 5.5 kHz, up to 5 kHz or both of the foregoing.

14. The intermediate storage unit as claimed in claim 10, further comprising a capacitor, a battery or both.

15. The intermediate storage unit as claimed in claims 10, wherein a cyclical provision of energy is effected for a load element having a cyclical power demand.

16. The intermediate storage unit as claimed in claim 10, wherein the load element comprises a beam offset unit, including a scanner and a scanner controller.

17. An ophthalmological laser therapy system with a power consumption of up to 1600 W, comprising an electrical load element and an intermediate storage unit configured to cyclically provide an energy of at least 30 mJ, for the electrical load element.

18. The ophthalmological laser therapy system as claimed in claim 17, wherein the ophthalmological laser therapy system comprises at least one further electrical load element and an energy supply unit, the energy supply unit being electrically connected to the electrical load element and the at least one further electrical load element in such a way that a supply with energy is made possible, and the intermediate storage unit being electrically connected to the energy supply unit and the electrical load element in such a way that the energy is provided cyclically only for the electrical load element.

19. The ophthalmological laser therapy system as claimed in claim 17, wherein the ophthalmological laser therapy system further comprises an energy supply unit configured as a wide-range power supply unit, and an interchangeable power supply system cable, via which the energy supply unit is electrically connectable to a local power supply system.

20. The ophthalmological laser therapy system as claimed in claim 17, wherein the intermediate storage unit is configured to cyclically provide an energy of at least 40 mJ, or at least 50 mJ, for the electrical load element.

21. The ophthalmological laser therapy system as claimed in claim 17, wherein the intermediate storage unit is configured to cyclically provide an energy selected from a group consisting of up to 220 mJ, up to 200 mJ, up to 180 mJ, for the electrical load element.

22. The ophthalmological laser therapy system as claimed in claim 17, wherein the intermediate storage unit effects a cyclical provision at a frequency selected from a group consisting of at least 20 Hz, at least 40 Hz, and at least 60 Hz, effects the cyclical provision is at a frequency selected from a group consisting of up to 6 kHz, up to 5.5 kHz, up to 5 kHz or both of the foregoing.

23. The ophthalmological laser therapy system as claimed in claim 17, wherein the intermediate storage unit further comprises a capacitor, a battery or both.

24. The ophthalmological laser therapy system as claimed in claim 17, wherein the intermediate storage unit effects a cyclical provision of energy for a load element having a cyclical power demand.

25. The ophthalmological laser therapy system as claimed in claim 7, wherein the load clement comprises a beam offset unit, including a scanner and a scanner controller.
